(19) United States
(12) Patent Application Publication
(10) Pub. No.: US 2025/0352254 A1
(43) Pub. Date: Nov. 20, 2025

(54) DEVICE FOR MANIPULATING AN INTRAMEDULLARY ROD, COMPRISING AN ADAPTER PIECE, SET FOR PROVIDING SUCH A DEVICE, ADAPTER PIECE, AND ADAPTER SET

(71) Applicant: JABEZ MEDICAL GMBH & CO. KG, Glattbach (DE)

(72) Inventor: Siegbert Gregel, Edertal (DE)

(21) Appl. No.: 19/099,428

(22) PCT Filed: Jul. 28, 2023
(86) PCT No.: PCT/DE2023/100560

§ 371 Date: Jul. 10, 2025

(30) Foreign Application Priority Data
Jul. 30, 2022 (DE) 20 2022 104 342.6

(51) Int. Cl. A61B 17/88; A61B 17/00; A61B 17/72

(52) U.S. Cl. CPC A61B 17/8872; A61B 17/7258; A61B 2017/00367; A61B 2017/00477

(57) ABSTRACT

A device (200) for actuating an intramedullary nail (1) is proposed, which extends in the axial longitudinal direction (L) and comprises a proximal end (202) and a distal end (204), and comprises an actuating shaft (300, 600) which is associated with an actuating element (400, 700), and which comprises at its distal end (304, 604) an engagement section (306, 606) for engaging in, and rotating or retaining, a counterpart complementary thereto, wherein an adapter piece (500, 800) is arranged between the distal end (304, 604) of the actuating shaft (300, 600) and the distal end (204) of the device (200), which adapter piece (500, 800) is connected in a rotatably fixed manner to the engagement section (306, 606) of the actuating shaft (300, 600) at the distal end (304, 604) of the actuating shaft (300, 600), and which comprises, at the distal end (204) of the device (200), an engagement section (506, 806), which is complementary to an engagement section (32, 1032) of the anchor element (24, 1024) of an oppositely arrangeable intramedullary nail (1). Furthermore, the invention relates to an adapter piece (500, 800) and an adapter set, as well as a set for providing a device (200) according to the invention.

The invention relates firstly to a device for actuating an intramedullary nail, according to the preamble of claim 1.

According thereto, the invention relates to a device for actuating an intramedullary nail, wherein the intramedullary nail is adapted for internal stabilization and/or fixing of an elongate hollow body, in particular a bone, extends in an axial longitudinal direction and comprises a proximal end and a distal end, and comprises an elongate spring element which is wound in a helical shape starting from the proximal end and with respect to the longitudinal direction, wherein the radial extension of the wound spring element can be reduced by tensioning the spring element in the winding direction and can be increased by detensioning the spring element counter to the winding direction, wherein the proximal end of the intramedullary nail comprises a nail head, in which a proximal end of the spring element is anchored in an anchor element, wherein the anchor element comprises an engagement section for a device for actuating an intramedullary nail, and wherein the spring element can be tensioned and detensioned by engaging in and rotating of the anchor element with such a device, wherein the device for actuating an intramedullary nail extends in the axial longitudinal direction and comprises a proximal end and a distal end, and comprises an actuating shaft which is associated with an actuating element, and which comprises at its distal end an engagement section for engaging in, and rotating or retaining, a counterpart complementary thereto.

The invention further relates to an adapter piece according to claim 14, a set according to claim 15, comprising an above-mentioned device and an adapter piece, and an adapter set according to claim 16.

Techniques and devices for intramedullary nailing in case of bone fractures of the extremities were first significantly developed and described by Küntscher et al., see Küntscher, G., Maatz, R., “Technik der Marknagelung”, Georg Thieme Verlag Leipzig, 1945.

Simpler intramedullary nails for interlocking nailing are known, but their installation during surgery can only be checked by means of intraoperative X-ray imaging. This is, due to the duration, stressful not only for the patient but in particular for the surgical staff. Furthermore, such simple intramedullary nails must be secured in the bone using transverse screws, which requires complex targeting devices, in particular for long femurs, but also for humeri and tibiae, in order to be able to place such transverse screws precisely through the skin with as little damage to the skin as possible. These additional surgical wounds that have to be inflicted into an extremity have to be treated after surgery, but also after surgical removal of the transverse screws, which causes additional stress for the patient.

WO 2006/066536 A1 discloses an intramedullary nail, in particular for the closed reposition of a fracture of a tubular bone by insertion into a drill hole in its medullary cavity, which is of comparatively simple construction and can be used easily, quickly and safely with only minimal use of intraoperative X-rays, without requiring interventions in unhurt areas of an extremity. The intramedullary nail comprises a nail core, at the front region of which a head piece is provided, at which the origin of a strip is fixed. The strip is wound around the nail core and is fixed at its end in the rear region around the nail core in a rotatably lockable manner, and the strip expands the radial contour of the intramedullary nail when it is rotated in the opposite direction to the direction in which it is wound.

Advantageous further developments of this intramedullary nail with a nail core are the subject of publications WO 2014/060576 A1 and WO 2014/060578 A1.

WO 2015/117996 A1 discloses a further intramedullary nail, namely an assembly for the internal stabilization and/or fixing of an elongate hollow body, in particular a bone, such as a tubular bone, or a prosthesis, with a stabilizing element with an elongate first element comprising a helical shape, the proximal end of which is connected to a first fastening element, and from the distal end of which a second element extends, which is connected to a second fastening element in the region of the first fastening element, wherein the first element and the second element preferably merge into one another connectionless as sections of the stabilizing element, wherein an outer fastening element forming the first fastening element clampingly fixes an inner fastening element forming the second fastening element, so that the first fastening element cannot be twisted relative to the second fastening element.

Other comparable intramedullary nails are shown in DE 10 2018 005 690 A1 and DE 20 2020 003 051 U1.

A tool, which is presently referred to as a device for actuating an intramedullary nail, is required for tensioning a spring element of such an intramedullary nail, namely for reducing its cross-section in order to be able to insert it into the medullary cavity, and for detensioning the spring element in order to be able to clamp the spring element in the inserted state with respect to the inner surface of the medullary cavity, and not least also for handling the intramedullary nail both during implantation and during explantation.

DE 20 2007 008 234 U1 discloses a mounting instrument for an intramedullary nail comprising at least one spiral spring body which can be expanded within the intramedullary nail channel by a relative rotatory movement. On the mounting instrument there is provided a handle for a positioning element and at least one rotary handle which is connected to a spiral spring body rotary element tube which can be rotated relative to the positioning element. This publication discloses a device of the kind mentioned at the beginning.

WO 2015/117996 A1, already mentioned in relation to an intramedullary nail, also shows a device for opposing rotation of two, in particular helical, elements of an assembly for internal stabilization, wherein the device comprises a holding section with a first hollow cylinder section, preferably with a first handle, wherein the first hollow cylinder section coaxially surrounds a second hollow cylinder section and a shaft-shaped or hollow cylinder-shaped inner section. This publication also shows a device of the kind mentioned at the beginning.

A disadvantage of the known devices for actuating an intramedullary nail lies in the insufficient variability. When using an intramedullary nail implant with a defined diameter, exactly the right device must be used. However, there are a large number of intramedullary nails with different diameters, depending on the age and gender of the patient and, in particular, on the type of bone to be treated. This results in a time-consuming, expensive and ultimately uneconomical process, because up to now, if a different diameter of intramedullary nail is required, a completely different device must be used to actuate the intramedullary nail. Either a large number of different devices must therefore be purchased and kept in stock, or the device currently required is not immediately available. An interaction of known devices for actuating an intramedullary nail with different intramedullary nails of different diameters is not yet possible.

The present invention is based on the problem of overcoming the disadvantages resulting from the state of the art.

Furthermore, the problem is to provide a device for actuating an intramedullary nail with which different intramedullary nails of different sizes or diameters may be actuated.

This problem is solved by a device of the kind mentioned at the beginning, which is further developed by the characterizing features of claim 1.

An adapter piece according to the invention is the subject of claim 14.

A set according to the invention comprising a device as mentioned at the beginning and an adapter piece according to the invention is the subject of claim 15.

A set according to the invention comprising a first outer adapter piece and a second inner adapter piece is the subject of claim 16.

Advantageous embodiments and further developments of the invention are the subject of the dependent claims.

According to claim 1, the generic device is designed and further developed in such a way that an adapter piece is arranged between the distal end of the actuating shaft and the distal end of the device, which adapter piece is connected in a rotatably fixed manner to the engagement section of the actuating shaft at the distal end of the actuating shaft, and which comprises, at the distal end of the device, an engagement section which is complementary to an engagement section of the anchor element of an oppositely arrangeable intramedullary nail.

The present invention thus provides a device for actuating an intramedullary nail which can be used for intramedullary nails, in particular double helix intramedullary nails, of different sizes. The device, which only has to be provided to the user once, can be adapted very quickly and inexpensively to any intramedullary nail geometry by quickly exchanging comparatively inexpensive adapter pieces.

This means that a wide range of important variants are available during surgical treatment. If it turns out during an operation that a model of a different size needs to be used instead of an originally planned intramedullary nail, the already available and appropriately prepared (in particular sterilized) device for actuating an intramedullary nail can be very quickly converted to a different adapter piece or set. In principle, this results in a high level of cost-effectiveness, because only a single actuation device needs to be purchased, kept ready and prepared for surgery.

It also saves space on the operating table, which is urgently needed for other different instruments and tools, because only a single device for actuating an intramedullary nail needs to be available.

Furthermore, regardless of the possibility of adaptation to different intramedullary nail sizes, the adapter system as a connecting element between the device for actuating an intramedullary nail and the intramedullary nail itself can be easily and inexpensively exchanged and replaced in the event of wear without having to replace the entire device.

The device for actuating an intramedullary nail according to the invention is suitable, inter alia, for the intramedullary nails disclosed in the above-mentioned patent documents.

An intramedullary nail adapted to be actuated by the present device according to the invention is further described in the German utility model application 20 2021 106 260.6, meanwhile published as DE 20 2021 106 260 U1, of the present applicant. The content of this application is hereby incorporated by reference into the present application.

In the context of the present invention, the terms “proximal” and “distal” are always understood from the perspective of the person, resp. the surgeon, who actuates and handles an intramedullary nail.

In the following, the aspects according to the invention are discussed further, for which purpose reference is made in part to non-limiting advantageous embodiments and further developments of the invention. The features of advantageous further developments can be realized individually or in any combination, whereby further advantageous embodiments of the invention are created.

Throughout the present discussion of configurations, further developments, examples and embodiments of the devices according to the invention, methods of using the devices and uses of the devices according to the invention are also described. These methods and uses also expressly lie within the scope of the present invention.

Preferably, the or at least one actuating shaft of the device extends in a longitudinal direction and comprises a proximal end and a distal end.

Preferably, a central axis of an actuating shaft coincides with the longitudinal axis.

Preferably, the distal end of the or at least one adapter piece coincides with the distal end of the device. Thus, an intramedullary nail that can be arranged oppositely can be coupled directly with the engagement section of its anchor element to the complementary engagement section of the adapter piece, or vice versa.

In a particularly preferred manner, the device comprises two actuating shafts for indirectly actuating two spring elements of an intramedullary nail that can be arranged oppositely. In particular, the two spring elements of double helix intramedullary nails can be actuated or held independently of one other.

In an advantageous further development of the device, in each case an adapter piece is arranged between the distal end of each actuating shaft and the distal end of the device. This can relate to devices with one, two or more actuating shafts.

In a further advantageous manner, two actuating shafts can be arranged concentrically within the device.

In particular, it is preferred that a first, outer actuating shaft is designed as a sleeve, and a second, inner actuating shaft is designed as a shaft rotatably supported therein.

In an advantageous embodiment of the device according to the invention, the adapter piece or at least one of the existing adapter pieces extends in the longitudinal direction, and comprises a proximal end and a distal end. Preferably, all, in particular two, existing adapter pieces extend in this manner.

The central axis of an adapter piece can coincide with the longitudinal axis.

As already mentioned, two adapter pieces are preferably provided in the device, in particular wherein the two adapter pieces are arranged concentrically.

In an advantageous specific embodiment, a first, outer adapter piece is designed as a sleeve, and a second, inner adapter piece is designed as a shaft rotatably supported therein.

Preferably, the distal end of a second adapter piece is passed through the proximal end of a first adapter piece.

A first, outer adapter piece can preferably be designed with a widened, hollow cylinder-like proximal end. The bottom of the hollow cylinder can serve as a distal stop for a second, inner adapter piece.

Preferably, a second, inner adapter piece can be designed with a widened, cylindrical proximal end, wherein a widening of a first adapter piece serves as a stop for a widening of the second adapter piece. This prevents the inner adapter piece from falling out of the device in the distal direction.

For appropriate transmission of a rotary movement or torque from the actuating shaft or from one of the actuating shafts, the distal end of an actuating shaft can comprise a radially extending pin, and the proximal end of an adapter piece can further comprise a radially extending groove which can engage the pin in a rotatably fixed manner. Or, viewed the other way around, the pin can engage in the radially extending groove of the adapter piece in a manner comparable to the tip of a screwdriver.

Such a design is preferred in particular when pairing a second, inner actuating shaft with a second, inner adapter piece.

Specifically, the distal end of the actuating shaft can preferably be shaped like a hollow cylinder, and the pin can be arranged at the bottom of the hollow cylinder.

The pin can be inserted into two opposite holes in the wall of the hollow cylinder, extending above the bottom of the hollow cylinder.

Alternatively, in an inverted configuration, the proximal end of an adapter piece may comprise a radially extending pin, and the distal end of an actuating shaft may comprise a radially extending groove which engages the pin in a rotatably fixed manner.

In a further preferred embodiment, the distal end of the or at least one actuating shaft comprises an axially extending groove, and the proximal end of the or at least one adapter piece comprises an axially extending key which engages in the groove in a rotatably fixed manner.

Such a design is preferred in particular when pairing a first, outer actuating shaft with a first, outer adapter piece.

Alternatively, in an inverted configuration, the proximal end of an adapter piece may comprise an axially extending groove, and the distal end of an actuating shaft may comprise an axially extending key which engages in the groove in a rotatably fixed manner.

Preferably, an adapter piece can comprise an external thread at the distal end. Preferably, this is a first, outer adapter piece.

Particularly preferably, a threaded sleeve can be arranged on the external thread of an adapter piece, which threaded sleeve can be screwed over onto the nail head of an intramedullary nail that can be arranged on the opposite side. For this purpose, the nail head or the proximal end of a suitable intramedullary nail can also comprise a corresponding external thread.

In a particularly preferred further development, the distal end of an actuating shaft, in particular an outer actuating shaft, comprises an external thread.

In this context, an adapter piece, in particular an outer adapter piece, can preferably be fixed in or on the actuating shaft by a union nut screwed onto the distal end of the actuating shaft. In a further advantageous manner, such a union nut can in particular fix an adapted widening or shoulder of the adapter piece in the distal direction. In this way, an adapter piece, in particular an outer adapter piece, can be fixed to the device. This adapter piece can then in turn advantageously retain a second, inner adapter piece, in particular as already described above.

An actuating element of the device according to the invention can preferably be designed as a handwheel.

The center of an actuating element, in particular of a handwheel, preferably coincides with the longitudinal axis of the device.

In a further advantageous embodiment, two axially spaced-apart actuating elements are provided, wherein a distal actuating element is associated with an outer actuating shaft, and a proximal actuating element is associated with an inner, in particular coaxial, actuating shaft.

Preferably, the proximal actuating element with the inner actuating shaft is rotatable relative to the distal actuating element with the outer actuating shaft. Preferably, the inner actuating shaft acts on the inner spring element of a coupled double helix intramedullary nail.

In a further embodiment, a lock (ratchet function) can be settable for each desired direction of rotation. Within the mode of operation of a ratchet system, only one direction of rotation, i.e. tensioning or detensioning of the relevant spring element, can thereby be permitted. Such a ratchet function is particularly advantageous when tensioning a double helix spring, preferably via the inner spring element, so that it can then be inserted into the medullary canal with a reduced diameter. A ratchet function can reliably prevent the undesired detensioning of the spring.

A ratchet mechanism required for this purpose can preferably be arranged in the distal actuating element. The proximal actuating element can be rotated relative to the distal actuating element.

In a further advantageous embodiment of the device according to the invention, the engagement section at the distal end of an adapter piece comprises: axially extending recesses, protrusions, teeth, prongs, spikes, or a plurality thereof. In this respect, a securely acting positive fit can be produced with the corresponding engagement sections of a coupled intramedullary nail.

Preferably, the engagement section at the distal end of an adapter piece is formed within an external thread or integrally with an external thread of the adapter piece. The distal end of such an adapter piece can thus simultaneously serve to transmit a torque to the intramedullary nail as well as to axially couple an intramedullary nail by means of a threaded sleeve.

A threaded sleeve of an adapter piece and/or a union nut of an actuating shaft is or are particularly preferably coated, in particular with a titanium nitride coating. Such a coating prevents seizing or cold welding of adjacent parts.

Preferably, an adapter piece and an associated actuating shaft are made of different materials. This measure also serves to prevent seizing of corresponding adjacent parts. Preferably, different types of stainless steel are used for this purpose.

An adapter piece of the device according to the invention is preferably hardened in order to increase resilience and service life. However, when wear occurs, adapter pieces can be replaced easily and inexpensively, whereas conventional devices require extensive repairs or even complete replacement.

In a first preferred embodiment of the adapter set according to the invention, the adapter set additionally comprises a threaded sleeve for the distal end of the first adapter piece, and a union nut for the proximal end of the first adapter piece.

The set can preferably contain at least two sets of a first outer adapter piece and a second inner adapter piece, namely each with a different extension of the distal ends, for connection to intramedullary nails with different diameters. In this way, a device can be used to actuate a wide variety of intramedullary nails with different diameters with minimum effort.

A set can thus contain adapter pieces for connection to different intramedullary nails, e.g. adapter pieces for an intramedullary nail diameter of 6 mm, 7 mm, 8 mm, 9 mm, 10 mm, 11 mm, 12 mm, or a plurality of these, or all of these.

### DRAWINGS

Supplementary or in addition to the advantageous embodiments and further developments of the teaching already discussed, embodiments of devices, uses and methods according to the invention shown in the drawings to the extent of FIGS. 1 to 11 are explained in more detail below. However, the examples discussed with reference to the drawings do not limit the invention to the examples shown. In discussing the embodiments with reference to the drawing, generally preferred embodiments and further developments of the teaching are also shown.

Further developments of the advantageous embodiments described above with features of the embodiments described below, just like further developments of the embodiments described below with features of the embodiments described above, expressly form further advantageous embodiments of the invention, and thus form part of the present disclosure.

With regard to the following explanation of the figures, it should generally be noted that, for reasons of clarity, reference signs already shown in previous figures and already explained above in this respect have in some cases not been repeated in subsequent figures and/or are in some cases not explained again on the basis of these subsequent figures. For the explanation of such reference signs and the associated technical features, reference is therefore made in full to the respective description of the corresponding preceding figures in order to avoid repetition.

It is shown in:

FIG. 1 a perspective view of an intramedullary nail for the actuation of which the device according to the invention is adapted,

FIG. 2 a lateral sectional view of a preferred embodiment of the device according to the invention for actuating an intramedullary nail,

FIG. 3 the corresponding side view of the device from FIG. 2,

FIG. 4 at the top a lateral sectional view and at the bottom a top view of the first, outer actuating shaft of the device of FIG. 2 and FIG. 3,

FIG. 5 at the top a top view and at the bottom a side sectional view of the second, inner actuating shaft of the device from FIG. 2 and FIG. 3,

FIG. 6 at the top a top view and at the bottom a side sectional view of the first, outer adapter piece of the device from FIG. 2 and FIG. 3,

FIG. 7 a side view of the second, inner adapter piece of the device from FIG. 2 and FIG. 3,

FIG. 8 a perspective view of the device of FIG. 2 and FIG. 3, seen from the distal end,

FIG. 9a a detailed perspective view illustrating the approach and coupling of the distal end of the device according to the invention from FIGS. 2, 3 and 8, to the proximal end of a suitable intramedullary nail according to FIG. 1,

FIG. 9b a representation like in FIG. 9a, but after coupling of the parts,

FIG. 10 a perspective, laterally viewed representation of a set according to the invention for providing the device according to the invention from FIGS. 2, 3 and 8, and

FIG. 11 a schematic, side view of a device according to the invention with a set of adapter sets according to the invention for actuating intramedullary nails with different intramedullary nail diameters with the same device.

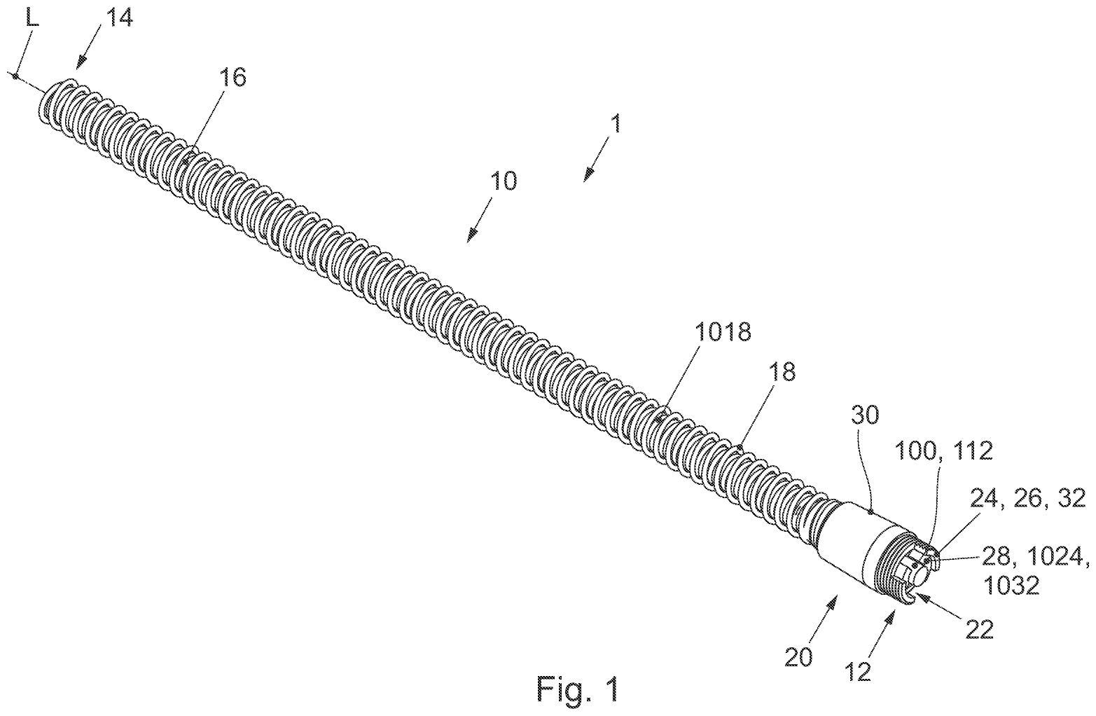

FIG. 1 shows a perspective view of an intramedullary nail 1, for the actuation of which the device according to the invention for actuating an intramedullary nail 1 is adapted. The illustration of this intramedullary nail 1 presently serves to provide a further understanding and illustration of the teaching according to the invention.

The intramedullary nail 1 shown is the subject of the German utility model application 20 2021 106 260.6, meanwhile published as DE 20 2021 106 260 U1, by the present applicant. The figures and figure description therein are hereby incorporated by reference into the present figure description.

The intramedullary nail 1 comprises a sheath element 10 and a nail core 100. The nail core 100 is of no further relevance in the present case, namely for the actuation of the intramedullary nail 1. In view of this fact, the sheath element 10 is also referred to below as intramedullary nail 1.

The intramedullary nail 1 is intended for the internal stabilization and/or fixing of an elongate hollow body, in particular a bone. It extends in a longitudinal direction L and comprises a proximal end 12 and a distal end 14.

The intramedullary nail 1 comprises a substantially cylindrical interior 16, wherein an outer contour of the intramedullary nail 1, viewed perpendicularly to the longitudinal direction L, comprises an elongate spring element 18 which, starting from the proximal end 12 and with respect to the longitudinal direction L, is wound in a helical shape.

The radial extension of the wound spring element 18 can be reduced by tensioning the elongate spring element 18 in the winding direction, and can be increased by detensioning the elongate spring element 18 counter to the winding direction.

Here, the spring element 18 essentially conceals the view on the interior 16, since the outer contour of the intramedullary nail 1 is essentially formed by the wound spring element 18. The spring element 18 is wound in the form of a three-dimensional coil, and here in particular in the form of a cylindrical coil.

The diameter of the intramedullary nail 1 can be reduced by tensioning the spring element 18. This is done in particular in order to be able to insert the intramedullary nail 1 into the medullary canal. On the other hand, the intramedullary nail 1 can also be braced, by expanding its outer contour, namely by detensioning the spring element 18, relative to the inner wall of an elongate hollow body to be stabilized, in particular in the medullary canal of a tubular bone.

In this embodiment, the intramedullary nail 1 comprises not just one spring element 18, but two elongate spring elements 18, 1018, arranged one above the other, which are each wound in a helical shape with respect to the longitudinal direction L of the intramedullary nail 1. For this purpose, a second spring element 1018 extends within the spring element 18 or first spring element 18.

Here, the two superimposed spring elements 18, 1018 form a three-dimensional double spiral, namely a double helix. The two spring elements 18, 1018 arranged one above the other are here wound in opposite directions compared to each other, and consist of one continuous element which merges, at the distal end 14, from the first spring element 18 into the second spring element 1018.

The proximal end 12 of the intramedullary nail 1 comprises a nail head 20. Both the proximal end of the first spring element 18 and the proximal end of the second spring element 1018 are anchored to the nail head 20. Furthermore, the nail head 20 comprises an axial aperture 22 for inserting the nail core 100.

The proximal end of the first spring element 18 is anchored in a first anchor element 24. The first anchor element 24 can also be referred to here as the outer anchor element 24, which is an outer sleeve 26.

The proximal end of the second spring element 1018 is anchored in a second anchor element 1024. The second anchor element 1024 can also be referred to here as the inner anchor element 1024, which is an inner sleeve 28.

The locking sleeve 30, which is arranged on the very outside, secures the anchoring of the outer, first spring element 18 to the first anchor element 24, i.e. to the outer sleeve 26. The locking sleeve 30 also conceals the further view of this section.

The inner sleeve 28 can be rotated relative to the outer sleeve 26. By twisting the inner sleeve 28 in the winding direction of the second spring element 1018, the spring element 1018 is tensioned and thus, with the outer sleeve 26 held in place or even twisted in the opposite direction, also tensions the first spring element 18 via the distal transition into the first spring element 18. The diameter of the intramedullary nail 1 is reduced.

By twisting the inner sleeve 28 against the winding direction of the second spring element 1018, the second spring element 1018 is detensioned and thus also detensions the first spring element 18 via the distal transition into the first spring element 18. For this purpose, it may already be sufficient to remove the previously applied torque in order to tension the second spring element 1018 from the inner sleeve 28. The second spring element 1018 may then already detension itself automatically, thereby rotating the inner sleeve 28 against the winding direction.

As a result, the outer spring element 18 can contact the inside of the medullary canal. Finally, the inner spring element 1018 may also contact the inside of the first spring element 18 in order to stabilize and secure it.

Alternatively or additionally, by twisting the outer sleeve 26, and thus the nail head 20, in the winding direction of the first spring element 18, the outer spring element 18 can be tensioned and thus the diameter of the intramedullary nail 1 may be reduced, and vice versa. The inner sleeve 28 should then be retained in place or even be actuated into the opposite direction.

In this illustration, the axial aperture 22 in the proximal end 12 of the intramedullary nail 1, namely in the nail head 20, can also be seen. Viewed from the inside out, the nail core 100 is surrounded by the inner sleeve 28, the outer sleeve 26, and the locking sleeve 30.

The design of the outer sleeve 26, which is recessed as viewed in the circumferential direction, provides a first engagement section 32 in the first anchor element 24. The design of the inner sleeve 28, which is recessed as viewed in the circumferential direction, provides a second engagement section 1032 in the second anchor element 1024. Both engagement sections 32, 1032 are formed in the form of axially extending teeth and provide useful engagement sections 32, 1032 for complementary engagement sections of an oppositely arrangeable device according to the invention for actuating an intramedullary nail 1.

Thus, for each spring element 18, 1018 in the intramedullary nail 1, a respective possibility for engagement is provided for a device according to the invention for actuating an intramedullary nail 1, namely for tensioning or detensioning the spring elements 18, 1018 of the intramedullary nail 1.

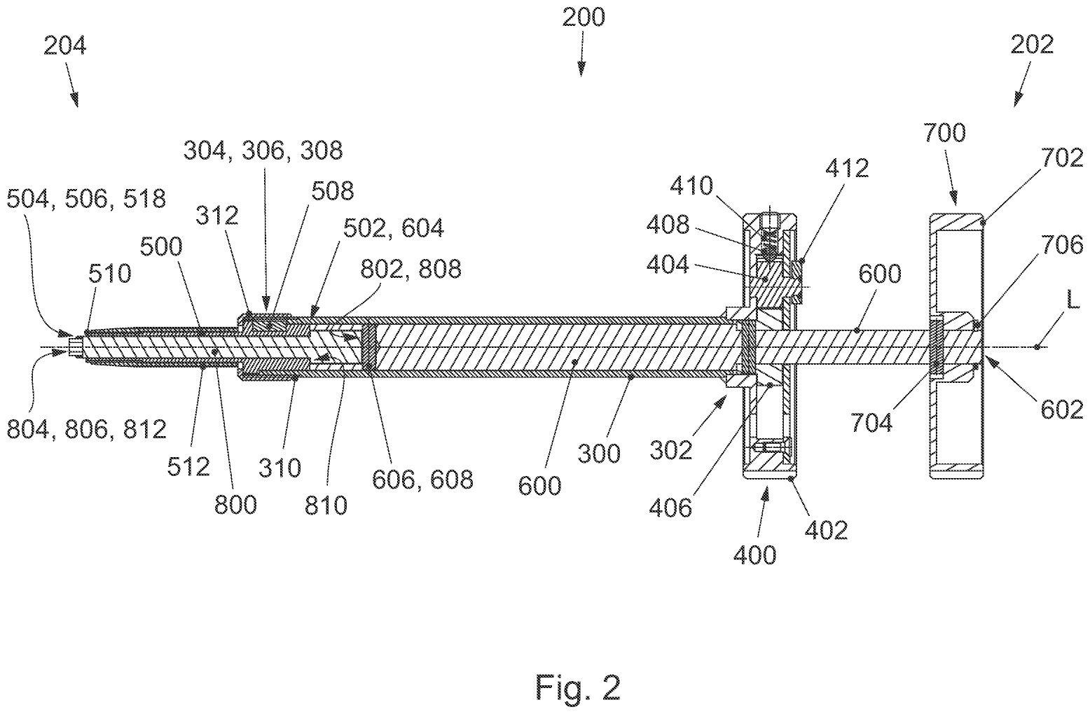

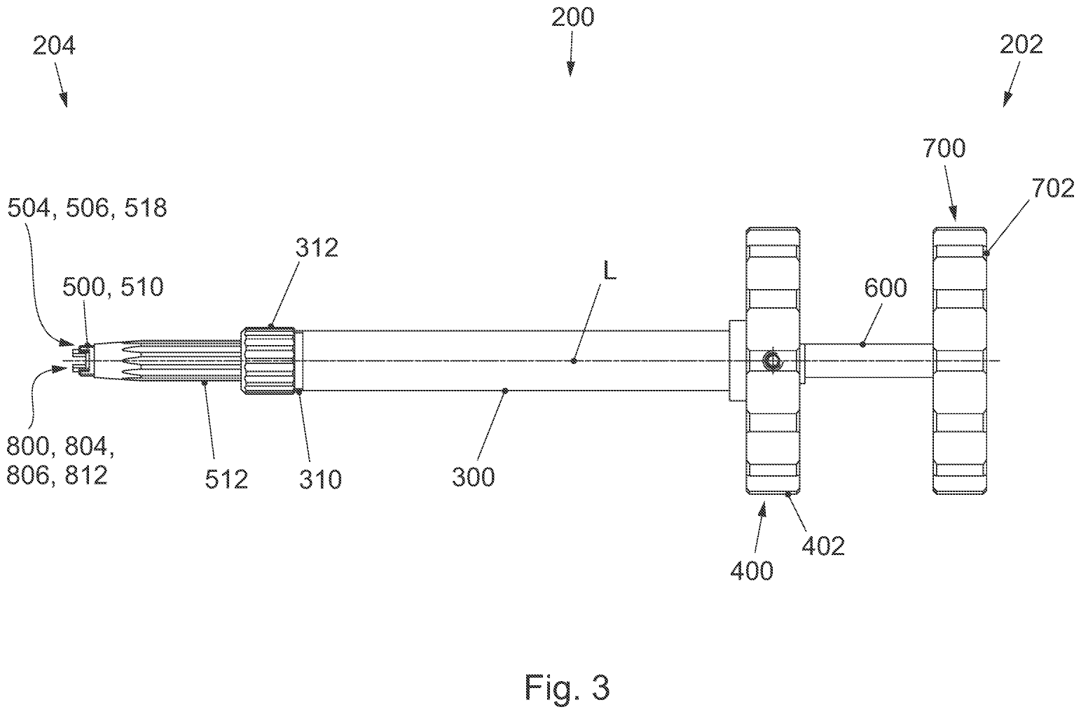

FIG. 2 shows a lateral sectional view of a preferred embodiment of the device 200 according to the invention for actuating an intramedullary nail 1, and FIG. 3 shows the corresponding lateral view of this device 200.

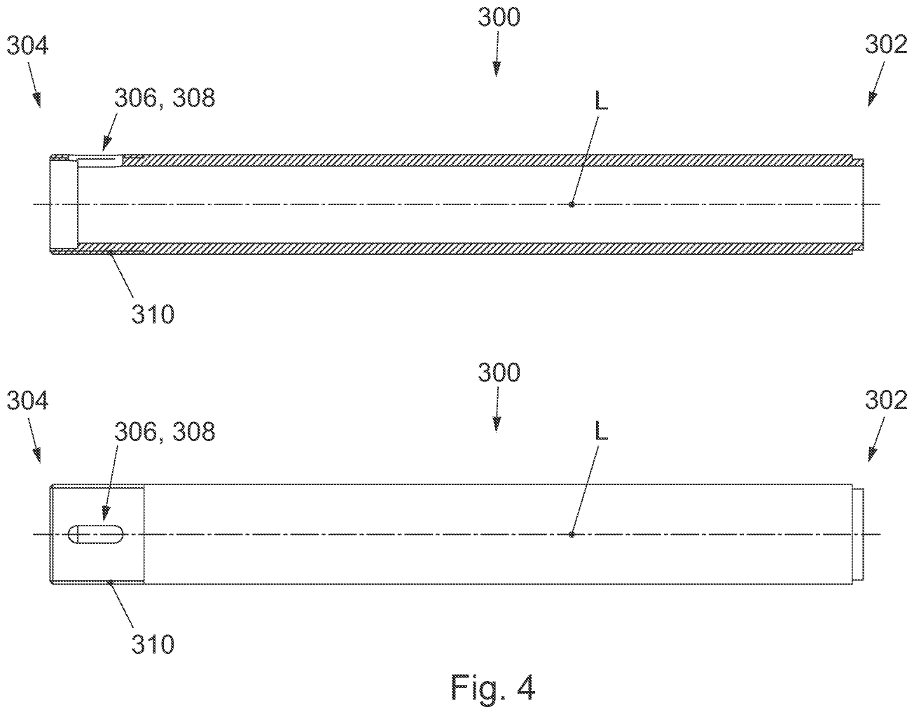

Concerning the reference numbers given below, supplementary reference is made to the individual illustrations in FIGS. 4 to 7.

The device 200 for actuating an intramedullary nail 1 extends in the axial longitudinal direction L and comprises a proximal end 202 and a distal end 204.

A first, outer actuating shaft 300 is associated with a first, distal actuating element 400. The outer actuating shaft 300 extends in the longitudinal direction L between a proximal end 302 and a distal end 304, and comprises at its distal end 304 an engagement section 306 for engaging and rotating, or retaining, a complementary counterpart.

A first, outer adapter piece 500 is arranged between the distal end 304 of the first, outer actuating shaft 300 and the distal end 204 of the device 200, which adapter piece 500 is, at the distal end 304 of the outer actuating shaft 300, non-rotatably connected to the engagement section 306 of the actuating shaft 300.

The adapter piece 500 comprises a proximal end 502 and a distal end 504, and extends therebetween in the longitudinal direction L, namely between the distal end 304 of the actuating shaft 300 and the distal end 204 of the device 200. The outer adapter piece 500 comprises, at the distal end 204 of the device 200, an engagement section 506, which is complementary to an engagement section 32 of the anchor element 24 of an intramedullary nail 1 that is arrangeable oppositely.

Here, the device 200 comprises two actuating shafts 300, 600 for indirectly actuating two spring elements 18, 1018 of an intramedullary nail 1 that can be arranged oppositely. The two actuating shafts 300, 600 are arranged concentrically.

A second, inner actuating shaft 600 is associated with a second, proximal actuating element 700. The inner actuating shaft 600 extends in the longitudinal direction L between a proximal end 602 and a distal end 604, and comprises at its distal end 604 an engagement section 606 for engaging in and rotating or retaining a complementary counterpart.

A second, inner adapter piece 800 is arranged between the distal end 604 of the second, inner actuating shaft 600 and the distal end 204 of the device 200, which, at the distal end 604 of the inner actuating shaft 600, is connected to the engagement section 606 of the actuating shaft 600 in a rotatably fixed manner.

The second, inner adapter piece 800 has a proximal end 802 and a distal end 804 and extends in the longitudinal direction L between the distal end 604 of the actuating shaft 600 and the distal end 204 of the device 200. The inner adapter piece 800 comprises, at the distal end 204 of the device 200, an engagement section 806, which is complementary to an engagement section 1032 of the anchor element 1024 of an intramedullary nail 1 that can be arranged oppositely.

Thus, an adapter piece 500, 800 is arranged here between the distal end 304, 604 of each actuating shaft 300, 600 and the distal end 204 of the device 200. Thus, two adapter pieces 500, 800 are provided here, wherein the adapter pieces 500, 800 are arranged concentrically.

The first, outer actuating shaft 300 is formed as a sleeve, and the second, inner actuating shaft 600 is formed as a shaft rotatably supported therein. Further, the first, outer adapter piece 500 is formed as a sleeve, and the second, inner adapter piece 800 is formed as a shaft rotatably supported therein.

The distal ends 504, 804 of the adapter pieces 500, 800 with their respective engagement sections 506, 806 thereby substantially coincide with the distal end 204 of the device 200.

Each engagement section 506, 806 of the adapter pieces 500, 800 thereby comprises axially extending recesses or protrusions, which may also be referred to as teeth 518, 812.

The respective engagement between the actuating shafts 300, 600 and the respectively associated adapter piece 500, 800 is explained below.

The distal end 604 of the second, inner actuating shaft 600 is provided with a radially extending pin 608. On the other hand, the opposite proximal end 802 of the second, inner adapter piece 800, comprises a radially extending groove 808, which can engage the pin 608 in a rotatably fixed manner. During assembly, the second adapter piece 800 can thus be pushed onto the distal end 604 of the second, inner actuating shaft 600, or vice versa, and the groove 808 engages around the pin 608, whereby a non-rotatable but axially releasable coupling is established between the inner actuating shaft 600 and the inner adapter piece 800.

The distal end 304 of the first, outer actuating shaft 300, on the other hand, comprises an axially extending groove 308 as an engagement section 306, and the proximal end 502 of the first, outer adapter piece 500 comprises an axially extending key 508, which engages in the groove 308 in a rotatably fixed manner.

The assembly comprising the inner actuating shaft 600 and the inner adapter piece 800 remains rotatable relative to the assembly comprising the outer actuating shaft 300 and the outer adapter piece 500. For assembly, the inner adapter piece 800 is inserted with its distal end 804 through the proximal end 502 of the outer adapter piece. Then, this assembly is inserted into the distal end 304 of the outer actuator shaft 300, so that the couplings of the key 508 and groove 308 and the groove 808 and pin 608 are established.

The distal end 304 of the first, outer actuating shaft 300 further comprises an external thread 310, and after insertion into the distal end 304, the two adapter pieces 500, 800 can be fixed in or on the actuating shaft 300 by the union nut 312 screwed onto the external thread 310.

Furthermore, the outer adapter piece 500 also comprises at its distal end 504 an external thread 510 in addition to the engagement section 506. A threaded sleeve 512 is screwed onto the external thread 510. In a particularly advantageous manner, the threaded sleeve 512 can be screwed onto a matching external thread in the nail head 20 or an outer sleeve 26 of an intramedullary nail 1 when coupling an intramedullary nail 1 to the device 200. In this way, not only can the spring element 18, 1018 of the intramedullary nail 1 be actuated with the device 200, but the intramedullary nail 1 can also be handled in a particularly advantageous manner with the device 200 during implantation or explantation, since it can be securely and firmly fixed to the device 200 with respect to the axial direction L.

The first, distal actuating element 400 is formed as a handwheel 402, and the second, proximal actuating element 700 is formed as a handwheel 702 spaced therefrom. The center of each handwheel 402, 702 coincides with the longitudinal axis L.

The first, distal actuating element 400 is firmly connected to the first, outer actuating shaft 300. The first actuating element 400 is used to hold the first actuating shaft 300 in place or to rotate it if necessary.

The second, proximal actuating element 700 is connected to the second, inner actuating shaft 600 via the pin 704 and the retaining ring 706. The second actuating element 700 is used to rotate the second actuating shaft 600 relative to the first actuating shaft 300 or, if necessary, to hold it in place.

For said rotatability of the second actuating shaft 600 relative to the first actuating shaft 300, a ratchet mechanism is implemented in the distal handwheel 402, whereby one direction of rotation can be locked in each case in the sense of a ratchet mechanism. Here, a ratchet 404 acts on a pinion 406. In the desired direction of rotation, the ratchet slides on the teeth of the pinion 406, but in the opposite direction, the ratchet 404 jams in the teeth and locks the rotation. Such a mechanism is known in itself.

The ratchet 404 is subjected to the force of a compression spring 410 via a ball 408. The respective direction of rotation can be set via a ratchet lever 412 by pivoting the ratchet 404 about the longitudinal axis L. Depending on the inclined positioning of the ratchet 404, it will lock the respective backward rotation of the inner actuating shaft 600.

FIG. 4 shows at the top a lateral sectional view and at the bottom a top view of the first, outer actuating shaft 300 of the device 200 of FIG. 2 and FIG. 3.

The outer actuating shaft 300 extends in the longitudinal direction L and comprises a proximal end 302 and a distal end 304. The central axis of the actuating shaft 300 coincides with the longitudinal axis L.

It can be clearly seen here that the outer actuating shaft 300 is designed as a sleeve in which a second, inner actuating shaft 600 can rotate as a shaft.

At the distal end 304 of the outer actuating shaft 300, the engagement section 306 with the axially extending groove 308 for the key 508 of the first adapter piece 500 can be clearly seen here.

On the other hand, the external thread 310, onto which the union nut 312 can be screwed, can be clearly seen here.

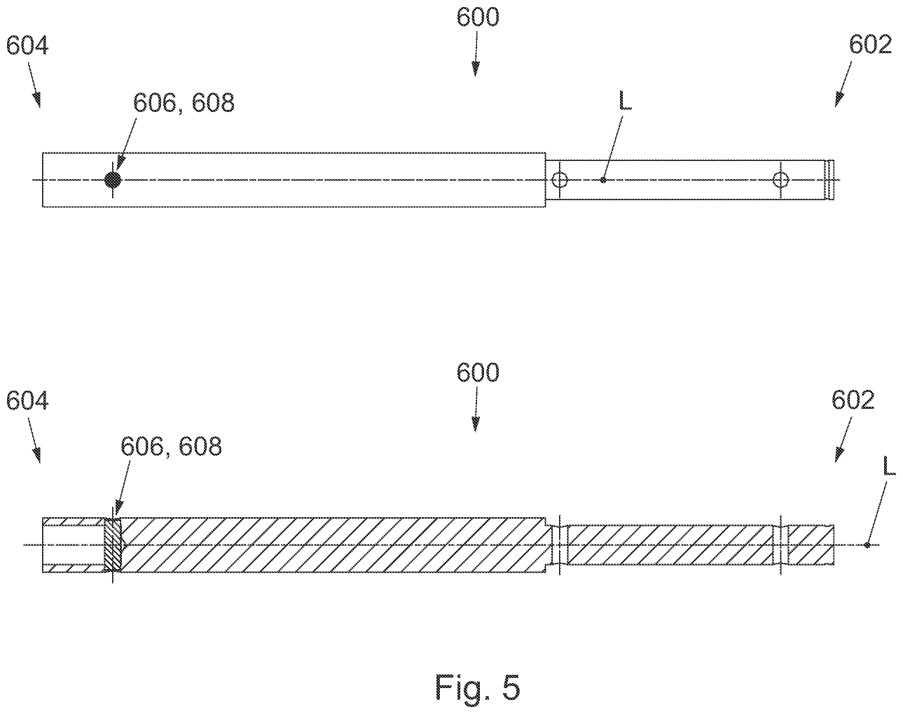

FIG. 5 shows at the top a top view and at the bottom a side sectional view of the second, inner actuating shaft 600 of the device 200 of FIG. 2 and FIG. 3.

The inner actuating shaft 600 extends in the longitudinal direction L and comprises a proximal end 602 and a distal end 604. The central axis of the actuating shaft 600 coincides with the longitudinal axis L.

The inner actuating shaft 600 is here designed as a shaft which can be inserted into the sleeve-like first actuating shaft 300 and can rotate there.

The distal end 604 of the inner actuating shaft 600 is shaped like a hollow cylinder. A radially extending pin 608 is arranged in the engagement section 606 of the inner actuating shaft 600, which is inserted into this hollow cylinder through two lateral bores and extends above the bottom of the hollow cylinder.

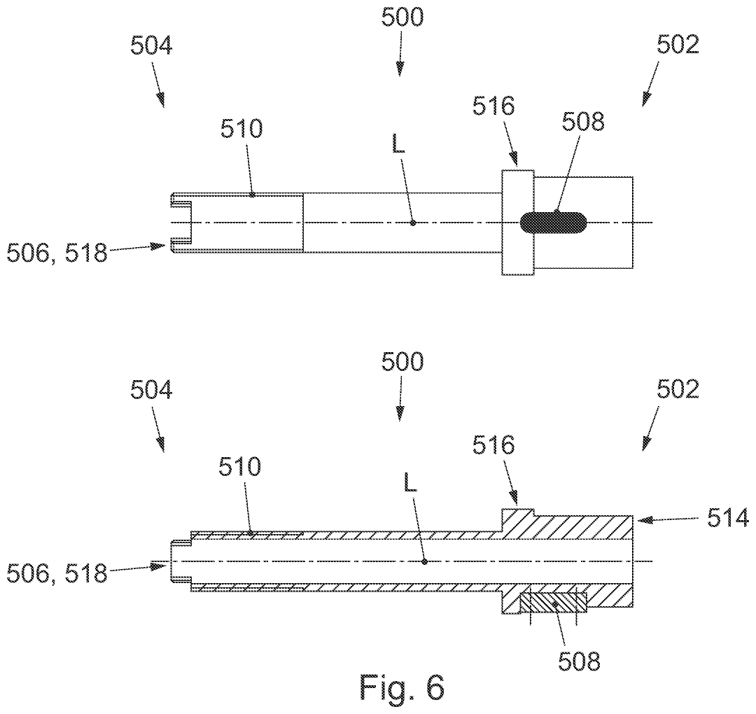

FIG. 6 shows at the top a top view and at the bottom a side sectional view of the first, outer adapter piece 500 of the device 200 of FIG. 2 and FIG. 3.

The outer adapter piece 500 extends in the longitudinal direction L and comprises a proximal end 502 and a distal end 504. The central axis of the adapter piece 500 coincides with the longitudinal axis L.

It can be clearly seen here that the outer adapter piece 500 is designed as a sleeve in which a second, inner adapter piece 800 can rotate as a shaft.

At the proximal end 502 of the outer adapter piece 500 there is on the one hand a widening 514 of the wall thickness. Thus, one can speak of a widened, hollow cylinder-like proximal end 502. The widening 514 serves on the one hand as a stop for the widening of the inner adapter piece 800 at its proximal end 802, and on the other hand as a stop for the distal end 604 of the inner actuating shaft 600.

On the other hand, a further widening, namely the collar 516, of the outer adapter piece 500 serves as a stop at the distal end 304 of the outer actuating shaft 300, and serves as a stop for the union nut 312, which is screwed onto the distal end 304 of the outer actuating shaft 300.

The key 508 inserted in the widening 514 serves to engage in the engagement section 306 (groove 308) of the first actuating shaft 300.

At the distal end 504, on the one hand the external thread 510 can be seen, which is used to screw on the threaded sleeve 512. On the other hand, the engagement section 506 for actuating the intramedullary nail 1 can be seen here, in which axially extending cylindrical segment-like teeth 518 are provided within the external thread 510.

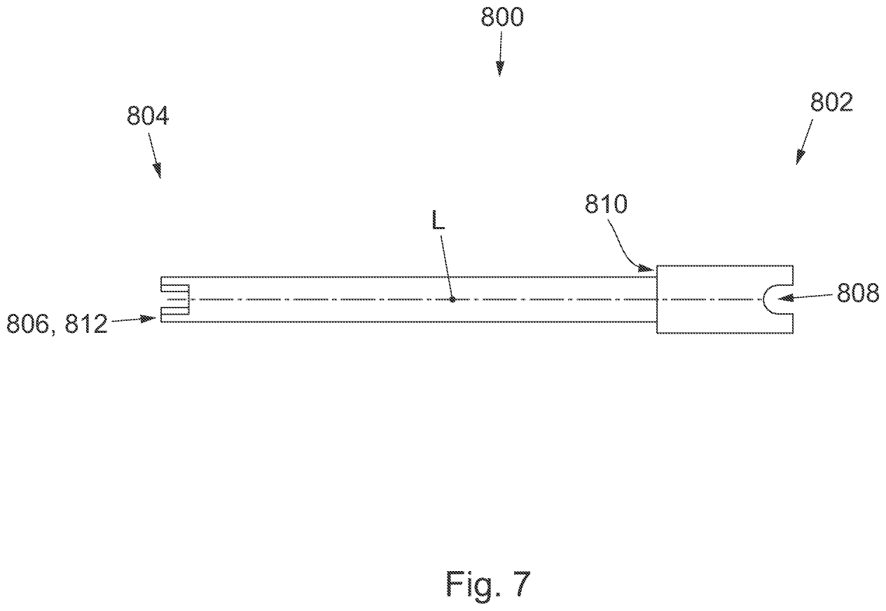

FIG. 7 shows a lateral view of the second, inner adapter piece 800 of the device 200 of FIG. 2 and FIG. 3.

The inner adapter piece 800 extends in the longitudinal direction L and comprises a proximal end 802 and a distal end 804. The central axis of the adapter piece 800 coincides with the longitudinal axis L.

It can be clearly seen here that the inner adapter piece 800 is designed as a shaft and can rotate in the first, outer adapter piece 500.

The inner adapter piece 800 comprises a widening 810 at the proximal end 802. Thus, one can speak of a widened, cylindrical proximal end 802. The widening 810 serves as a stop relative to the proximal end 502 of the outer adapter piece 500.

Furthermore, the radially extending groove 808 is formed at the proximal end 802, which serves to engage the engagement section 606, namely the pin 608, of the inner actuating shaft 600.

At the distal end 804, the engagement section 806 is formed for engagement with the intramedullary nail 1, and consists of axially extending teeth 812.

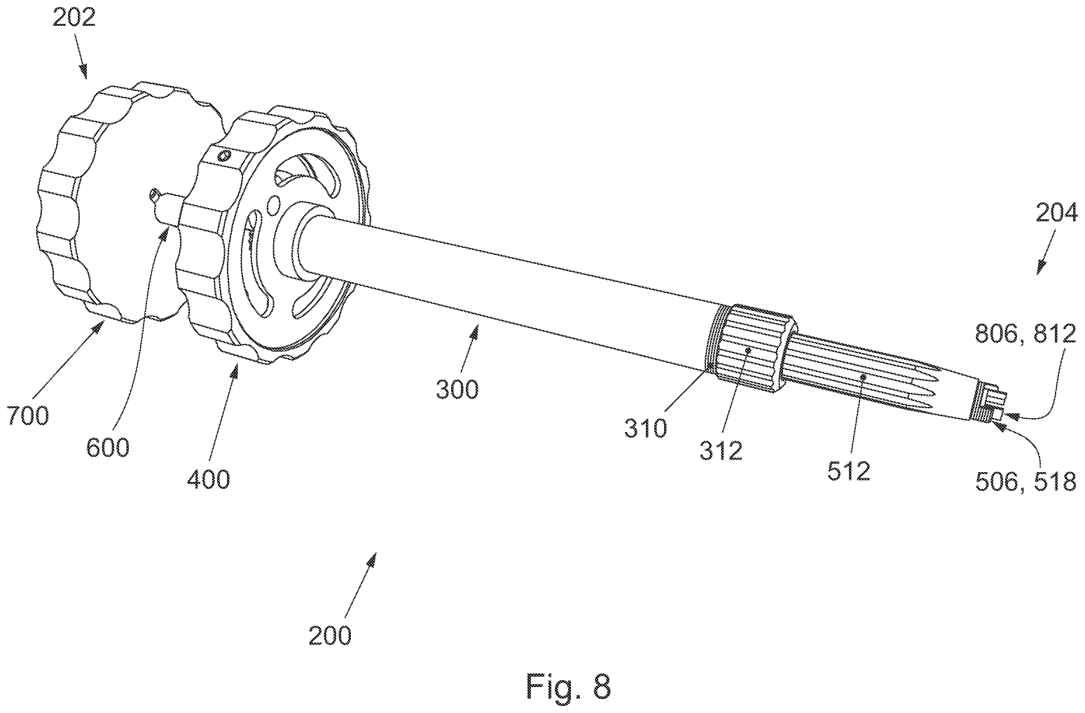

FIG. 8 shows a perspective view of the device 200 of FIG. 2 and FIG. 3 as seen from the distal end 204. The following can be seen from the outside (starting from the proximal end 202 to the distal end 204): proximal actuating element 700, inner actuating shaft 600, distal actuating element 400, outer actuating shaft 300, external thread 310, union nut 312, threaded sleeve 512, engagement section 506 on the outside with teeth 518, engagement section 806 on the inside with teeth 812.

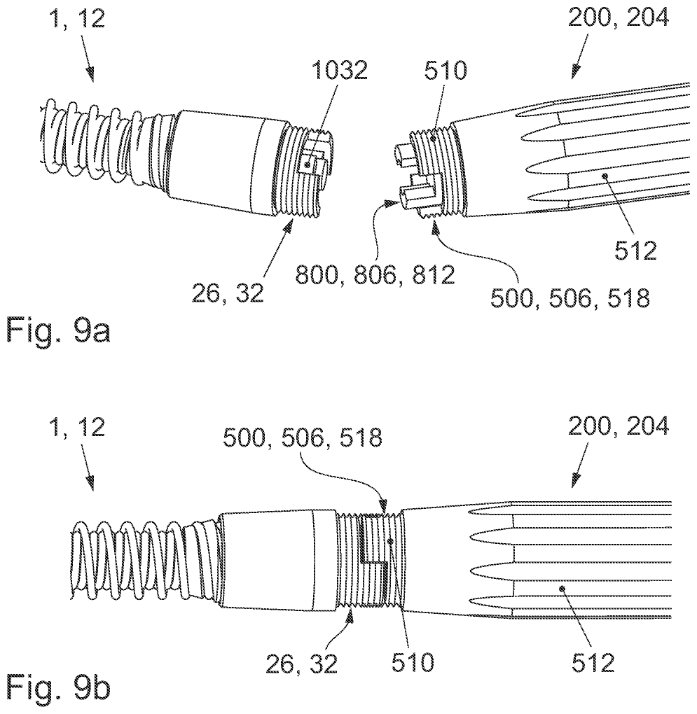

FIG. 9a shows a detailed perspective view illustrating the approach and coupling of the distal end 204 of the device 200 according to the invention of FIGS. 2, 3 and 8 to the proximal end 12 of a suitable intramedullary nail 1 according to FIG. 1, and FIG. 9b shows a corresponding view after coupling.

The outer engagement section 506 of the outer adapter piece 500 is brought to the outer engagement section 32 of the intramedullary nail 1, and the inner engagement section 806 of the inner adapter piece 800 is brought to the inner engagement section 1032 of the intramedullary nail 1.

The inner teeth 812 engage in the inner engagement section 1032. The outer teeth 518 are formed as segment-like recesses or teeth 518 in the outer thread 510, and the outer engagement section 32 of the intramedullary nail 1 is formed exactly complementary thereto. Thus, the teeth 518 can interlock with the outer sleeve 26 of the intramedullary nail 1, and at the same time a continuous outer thread 510 is formed, onto which the threaded sleeve 512 can then be screwed. The intramedullary nail 1 is thereby fixed axially and can then be actuated and handled by the device 200.

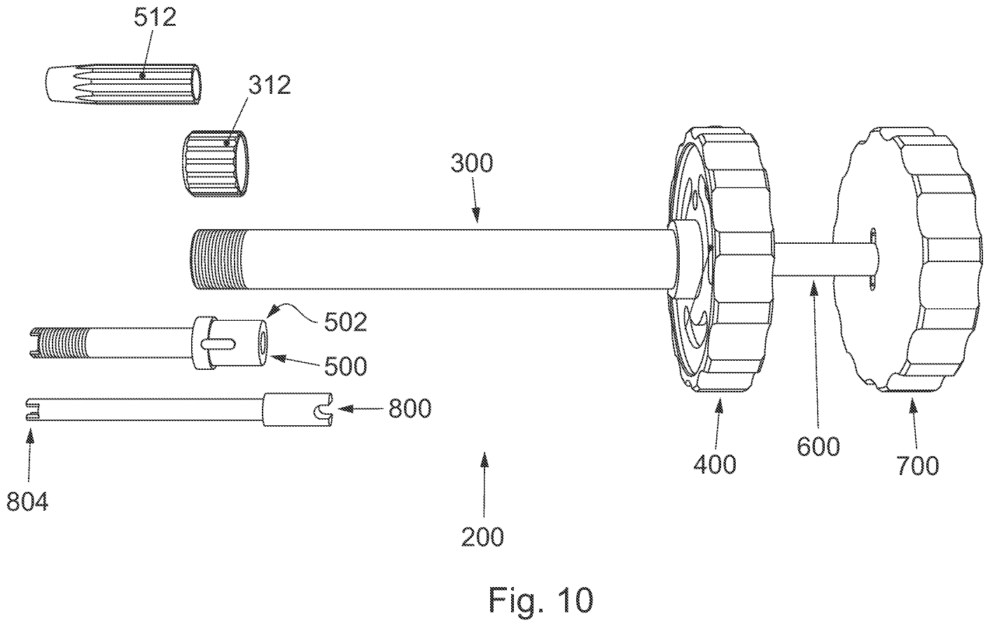

FIG. 10 shows a perspective view, seen from the side, of a set according to the invention for providing the device 200 according to the invention from FIGS. 2, 3 and 8. The set contains at least one device 200 comprising the distal actuating element 400, the proximal actuating element 700, the outer actuating shaft 300, the inner actuating shaft 600, as well as at least one adapter piece 500, 800, preferably with both adapter pieces, the outer adapter piece 500 and the inner adapter piece 800, particularly preferably additionally with the union nut 312 and the threaded sleeve 512.

In this representation of the individual parts, it is also particularly easy to see how the distal end 804 of the second, inner adapter piece 800 can be passed through the proximal end 502 of the first, outer adapter piece 500 during assembly.

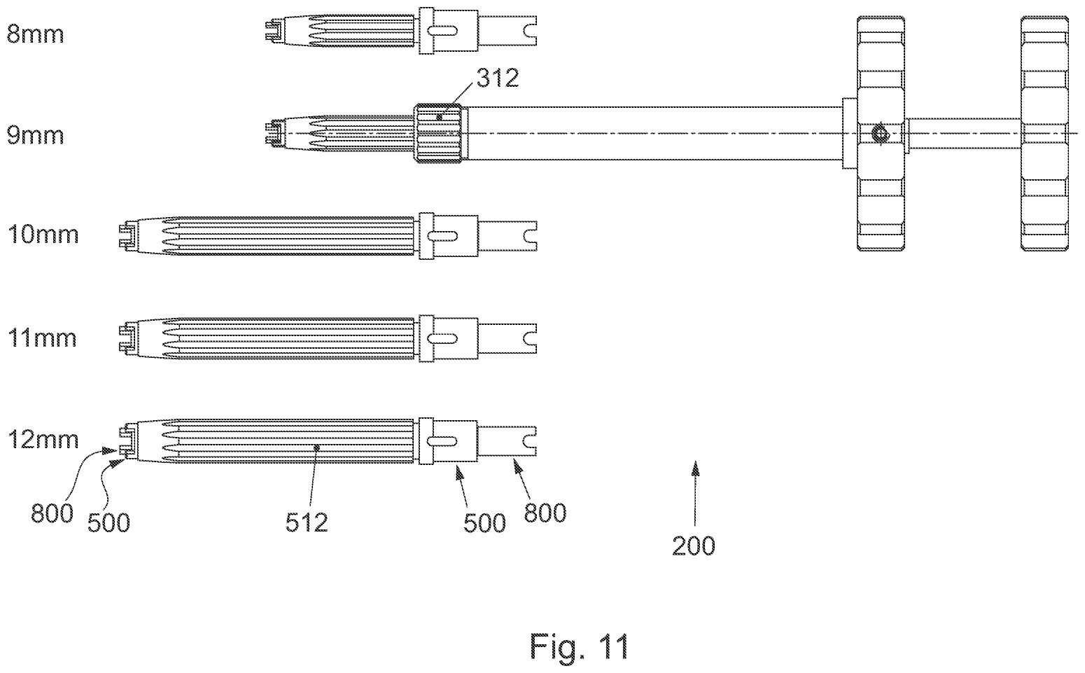

FIG. 11 shows a schematic, lateral representation of a device 200 according to the invention with a set of adapter sets according to the invention for actuating intramedullary nails 1 with different intramedullary nail diameters with the same device 200.

An adapter set comprises at least the first, outer adapter piece 500 and the inner adapter piece 800, which are each adapted to the diameter of the intramedullary nail 1 to be connected. Preferably, an adapter set can also comprise adapted threaded sleeves 512 and union nuts 312.

Adapter sets for connecting intramedullary nails 1 with a diameter of 8, 9, 10, 11 or 12 mm are shown schematically. It becomes clear that intramedullary nails 1 with a wide variety of diameters can be actuated with the device 200 according to the invention, whereby the effort for procuring suitable adapter sets is practically negligible compared to procuring different devices 200.

### EMBODIMENTS

Supplementary or in addition to the advantageous embodiments, further developments and examples already discussed, the invention is described below with reference to further specific preferred embodiments, which, however, do not limit the invention to the embodiments described. These embodiments are expressly part of the present description.

Further developments of the advantageous embodiments and examples described above with features of the embodiments described below, just like further developments of the embodiments described below with features of the embodiments and examples described above, expressly form further advantageous embodiments of the invention, and thus form part of the present disclosure.
Embodiment 1: Device for actuating an intramedullary nail, wherein the intramedullary nail is adapted for internal stabilization and/or fixing of an elongate hollow body, in particular a bone, extends in an axial longitudinal direction and comprises a proximal end and a distal end, and comprises an elongate spring element which is wound in a helical shape starting from the proximal end and with respect to the longitudinal direction, wherein the radial extension of the wound spring element can be reduced by tensioning the spring element in the winding direction and can be increased by detensioning the spring element counter to the winding direction, wherein the proximal end of the intramedullary nail comprises a nail head, in which a proximal end of the spring element is anchored in an anchor element, wherein the anchor element comprises an engagement section for a device for actuating an intramedullary nail, and wherein the spring element can be tensioned and detensioned by engaging in and rotating of the anchor element with such a device, wherein the device for actuating an intramedullary nail extends in the axial longitudinal direction and comprises a proximal end and a distal end, and comprises an actuating shaft which is associated with an actuating element, and which comprises at its distal end an engagement section for engaging in, and rotating or retaining, a counterpart complementary thereto.
Embodiment 2: Device according to the preceding embodiment, wherein an adapter piece is arranged between the distal end of the actuating shaft and the distal end of the device, which adapter piece is connected in a rotatably fixed manner to the engagement section of the actuating shaft at the distal end of the actuating shaft, and which comprises, at the distal end of the device, an engagement section, which is complementary to an engagement section of the anchor element of an oppositely arrangeable intramedullary nail.
Embodiment 3: Device according to one or both of the two preceding embodiments, wherein the actuating shaft extends in the longitudinal direction and comprises a proximal end and a distal end.
Embodiment 4: Device according to one or more of the preceding embodiments, wherein a central axis of the actuating shaft coincides with the longitudinal axis.
Embodiment 5: Device according to one or more of the preceding embodiments, wherein the distal end of the adapter piece coincides with the distal end of the device.
Embodiment 6: Device according to one or more of the preceding embodiments, wherein the device comprises two actuating shafts for indirectly actuating two spring elements of an intramedullary nail which is oppositely arrangeable.
Embodiment 7: Device according to one or more of the preceding embodiments, wherein in each case an adapter piece is arranged between the distal end of each actuating shaft and the distal end of the device.
Embodiment 8: Device according to one or more of the preceding embodiments, wherein two actuating shafts are concentrically arranged.
Embodiment 9: Device according to one or more of the preceding embodiments, wherein a first, outer actuating shaft is designed as a sleeve, and a second, inner actuating shaft is designed as a shaft rotatably supported therein.
Embodiment 10: Device according to one or more of the preceding embodiments, wherein the adapter piece extends in the longitudinal direction and comprises a proximal end and a distal end.
Embodiment 11: Device according to one or more of the preceding embodiments, wherein a central axis of the adapter piece coincides with the longitudinal axis.
Embodiment 12: Device according to one or more of the preceding embodiments, wherein two adapter pieces are provided, in particular wherein the two adapter pieces are arranged concentrically.
Embodiment 13: Device according to one or more of the preceding embodiments, wherein a first, outer adapter piece is designed as a sleeve, and a second, inner adapter piece is designed as a shaft rotatably supported therein.
Embodiment 14: Device according to one or more of the preceding embodiments, wherein the distal end of a second adapter piece is passed through the proximal end of a first adapter piece.
Embodiment 15: Device according to one or more of the preceding embodiments, wherein a first, outer adapter piece is designed with a widened, hollow cylinder-like proximal end.
Embodiment 16: Device according to one or more of the preceding embodiments, wherein a second, inner adapter piece is designed with a widened, cylindrical proximal end, wherein a widening of a first adapter piece serves as a stop for a widening of the second adapter piece.
Embodiment 17: Device according to one or more of the preceding embodiments, wherein the distal end of an actuating shaft comprises a radially extending pin, and the proximal end of an adapter piece comprises a radially extending groove which engages the pin in a rotatably fixed manner.
Embodiment 18: Device according to the preceding embodiment, wherein the distal end of the actuating shaft is hollow-cylindrical in shape, and the pin is arranged at the bottom of the hollow cylinder.
Embodiment 19: Device according to one or more of the preceding embodiments, wherein the proximal end of an adapter piece comprises a radially extending pin, and the distal end of an actuating shaft comprises a radially extending groove which engages the pin in a rotatably fixed manner.
Embodiment 20: Device according to one or more of the preceding embodiments, wherein the distal end of an actuating shaft comprises an axially extending groove, and the proximal end of an adapter piece comprises an axially extending key which engages in the groove in a rotatably fixed manner.
Embodiment 21: Device according to one or more of the preceding embodiments, wherein the proximal end of an adapter piece comprises an axially extending groove, and the distal end of an actuating shaft comprises an axially extending key which engages in the groove in a rotatably fixed manner.
Embodiment 22: Device according to one or more of the preceding embodiments, wherein an adapter piece comprises an external thread at the distal end.
Embodiment 23: Device according to one or more of the preceding embodiments, wherein a threaded sleeve is arranged on the external thread of an adapter piece, which sleeve can be screwed onto the nail head of an oppositely arrangeable intramedullary nail.
Embodiment 24: Device according to one or more of the preceding embodiments, wherein the distal end of an actuating shaft, in particular of an outer actuating shaft, comprises an external thread.
Embodiment 25: Device according to one or more of the preceding embodiments, wherein an adapter piece is fixed in or on the actuating shaft by a union nut screwed onto the distal end of the actuating shaft, in particular whereby the union nut fixes a suitable widening or shoulder of the adapter piece in the distal direction.
Embodiment 26: Device according to one or more of the preceding embodiments, wherein an actuating element is designed as a handwheel.
Embodiment 27: Device according to one or more of the preceding embodiments, wherein the center of an actuating element, in particular of a handwheel, coincides with the longitudinal axis.
Embodiment 28: Device according to one or more of the preceding embodiments, wherein two axially spaced apart actuating elements are provided, wherein a distal actuating element is associated with an outer actuating shaft, and a proximal actuating element is associated with an inner, in particular coaxial, actuating shaft.
Embodiment 29: Device according to the preceding embodiment, wherein the proximal actuating element together with the inner actuating shaft can be rotated relative to the distal actuating element together with the outer actuating shaft.
Embodiment 30: Device according to the preceding embodiment, wherein a lock (ratchet function) is settable for each desired direction of rotation.
Embodiment 31: Device according to the preceding embodiment, wherein a ratchet mechanism is arranged in the distal actuating element.
Embodiment 32: Device according to one or more of the preceding embodiments, wherein the engagement section at the distal end of an adapter piece comprises: axially extending recesses, protrusions, teeth, prongs, spikes, or a plurality thereof.
Embodiment 33: Device according to one or more of the preceding embodiments, wherein the engagement section at the distal end of an adapter piece is formed within an external thread or integral with an external thread of the adapter piece.
Embodiment 34: Device according to one or more of the preceding embodiments, wherein a threaded sleeve of an adapter piece and/or a union nut of an actuating shaft is or are coated, in particular with a titanium nitride coating.
Embodiment 35: Device according to one or more of the preceding embodiments, wherein an adapter piece and an associated actuating shaft are made of different material.
Embodiment 36: Device according to one or more of the preceding embodiments, wherein an adapter piece is hardened.
Embodiment 37: Adapter piece which is arrangeable between the distal end of the actuating shaft and the distal end of a device according to one or more of the preceding embodiments, and which, at the distal end of the actuating shaft of the device, is connectable in a rotatably fixed manner to the engagement section of the actuating shaft, and which comprises at its distal end an engagement section which is complementary to an engagement section of the anchor element of an intramedullary nail which is oppositely arrangeable.
Embodiment 38: Set comprising a device according to embodiment 1 and an adapter piece according to embodiment 37, for providing a device according to one or more of the preceding embodiments 2 to 36.
Embodiment 39: Set of a first outer adapter piece and a second inner adapter piece, each optionally according to embodiment 37, for providing a device according to one or more of the preceding embodiments 2 to 36.
Embodiment 40: Set according to the preceding embodiment, wherein the set additionally comprises a threaded sleeve for the distal end of the first adapter piece and a union nut for the proximal end of the first adapter piece.
Embodiment 41: Set according to one of the two preceding embodiments, wherein at least two sets of a first outer adapter piece and a second inner adapter piece are comprised, namely each with a different extension of the distal ends, for connecting to intramedullary nails with different diameters.
Embodiment 42: Set according to one or more of the three preceding embodiments, wherein in each case adapter pieces are included for connecting to intramedullary nails with a diameter of 6 mm, 7 mm, 8 mm, 9 mm, 10 mm, 11 mm, 12 mm, or a plurality of these, or all of these.

List of reference signs

| L | Longitudinal direction, length |
|---|---|
| 1 | Intramedullary nail |
| 10 | Sheath element |
| 12 | Proximal end (intramedullary nail) |
| 14 | Distal end (intramedullary nail) |
| 16 | Interior (intramedullary nail) |
| 18 | Spring element, first spring element, outer spring element |
| 1018 | Second spring element, inner spring element |
| 20 | Nail head |
| 22 | Axial aperture |
| 24 | First anchor element, outer anchor element |
| 1024 | Second anchor element, inner anchor element |
| 26 | Outer sleeve |
| 28 | Inner sleeve |
| 30 | Locking sleeve |
| 32 | First engagement section, outer engagement section |
| 1032 | Second engagement section, inner engagement section |
| 100 | Nail core |
| 200 | Device for actuating an intramedullary nail |
| 202 | Proximal end (device) |
| 204 | Distal end (device) |
| 300 | First actuating shaft, outer actuating shaft |
| 302 | Proximal end (first actuating shaft) |
| 304 | Distal end (first actuating shaft) |
| 306 | Engagement section (first actuating shaft) |
| 308 | Groove (first actuating shaft) |
| 310 | External thread (first actuating shaft) |
| 312 | Union nut |
| 400 | First actuating element, distal actuating element |
| 402 | Handwheel (first actuating element) |
| 404 | Ratchet |
| 406 | Pinion |
| 408 | Ball |
| 410 | Pressure spring |
| 412 | Ratchet lever |
| 500 | First adapter piece, outer adapter piece |
| 502 | Proximal end (first adapter piece) |
| 504 | Distal end (first adapter piece) |
| 506 | Engagement section (first adapter piece) |
| 508 | Key (first adapter piece) |
| 510 | External thread (first adapter piece) |
| 512 | Threaded sleeve |
| 514 | Widening (first adapter piece) |
| 516 | Collar (first adapter piece) |
| 518 | Tooth (first adapter piece) |
| 600 | Second actuating shaft, inner actuating shaft |
| 602 | Proximal end (second actuating shaft) |
| 604 | Distal end (second actuating shaft) |
| 606 | Engagement section (second actuating shaft) |
| 608 | Pin (second actuating shaft) |
| 700 | Second actuating element, proximal actuating element |
| 702 | Handwheel (second actuating element) |
| 704 | Pin |
| 706 | Circlip |
| 800 | Second adapter piece, inner adapter piece |
| 802 | Proximal end (second adapter piece) |
| 804 | Distal end (second adapter piece) |
| 806 | Engagement section (second adapter piece) |
| 808 | Groove (second adapter piece) |
| 810 | Widening (second adapter piece) |
| 812 | Tooth (second adapter piece) |

1.-16. (canceled)

17. A device configured for actuating an intramedullary nail and for extending in an axial longitudinal direction, the device comprising:
a proximal end;
a distal end;
an actuating shaft, which is associated with an actuating element, and which comprises at a distal end an engagement section configured for engaging in, and rotating or retaining, a complementary counterpart; and
an adapter piece arranged between the distal end of the actuating shaft and the distal end of the device,
wherein the adapter piece is connected in a rotatably fixed manner to the engagement section of the actuating shaft at the distal end of the actuating shaft,
wherein the adapter piece comprises, at the distal end of the device, an engagement section, which is complementary to an engagement section of an anchor element of the intramedullary nail, and
wherein the device is configured to tension and de-tension a spring element of the intramedullary nail by engaging in and rotating of the anchor element.

18. The device according to claim 17, wherein two actuating shafts configured for indirect actuation of two spring elements of the nails are provided.

19. The device according to claim 18, wherein, in each case, an adapter piece is arranged between the distal end of each actuating shaft and the distal end of the device.

20. The device according to claim 17, wherein the adapter piece extends in the longitudinal direction and comprises a proximal end and a distal end.

21. The device according to claim 17, wherein a central axis of the adapter piece coincides with the longitudinal direction.

22. The device according to claim 17, wherein a first outer adapter piece is a sleeve and a second inner adapter piece is a shaft rotatably supported therein.

23. The device according to claim 22, wherein the distal end of a second adapter piece is passed through the proximal end of the first outer adapter piece.

24. The device according to claim 17, wherein the distal end of at least one actuating shaft comprises a radially extending pin, and the proximal end of the adapter piece comprises a radially extending groove, which engages with the pin in a rotatably fixed manner.

25. The device according to claim 24, wherein the distal end of the actuating shaft is a hollow cylinder, and wherein the pin is arranged at a bottom of the hollow cylinder.

26. The device according to claim 17, wherein the distal end the actuating shaft comprises an axially extending groove and the proximal end of the adapter piece comprises an axially extending key, which engages in the groove in a rotatably fixed manner.

27. The device according to claim 17,
wherein the adapter piece comprises an external thread,
wherein a threaded sleeve is arranged on the external thread of the adapter piece, and wherein the threaded sleeve is screwable onto a nail head of the intramedullary nail.

28. The device according to claim 17, wherein the actuating shaft comprises an external thread, and
wherein the adapter piece is fixed in or on the actuating shaft by a union nut screwed onto the distal end of the actuating shaft.

29. The device according to claim 17, wherein the engagement section at the distal end of the adapter piece is formed within an external thread or is integral with the external thread of the adapter piece.

30. A system comprising:
an intramedullary nail extending in an axial longitudinal direction, the intramedullary comprising:
a proximal end;
a distal end;
an elongate spring element, which is wound in a helical shape with respect to the longitudinal direction starting from the proximal end,
wherein a radial extension of the wound spring element is reducible by tensioning the spring element in a winding direction and extendable by detensioning of the spring element,
wherein the proximal end comprises a nail head, in which a proximal end of the spring element is anchored in an anchor element, and
wherein the anchor element comprises an engagement section configured for actuating the intramedullary nail; and
a device configured for actuating the intramedullary nail and extending in the axial longitudinal direction, the device comprising:
a proximal end;
a distal end;
an actuating shaft, which is associated with an actuating element, and which comprises at a distal end an engagement section configured for engaging in, and rotating or retaining, a complementary counterpart; and
an adapter piece arranged between the distal end of the actuating shaft and the distal end of the device,
wherein the adapter piece is connected in a rotatably fixed manner to the engagement section of the actuating shaft at the distal end of the actuating shaft,
wherein the adapter piece comprises, at the distal end of the device, an engagement section, which is complementary to the engagement section of the anchor element of the intramedullary nail, and
wherein the spring element is tensionable and detensionable by engaging in and rotating of the anchor element with the device.

31. An adapter piece configured for actuating an intramedullary nail, the adapter piece comprising:
a proximal end and a distal end;
a first engagement section located at the proximal end configured to connect in a rotatably fixed manner to a complementary engagement section of an actuating shaft of a device for actuating the intramedullary nail; and
a second engagement section located at the distal end and complementary to an engagement section of an anchor element of the intramedullary nail,
wherein the adapter piece is configured to transmit rotational motion from the actuating shaft to the anchor element for tensioning and de-tensioning a spring element of the intramedullary nail.
